(19) United States
(12) Patent Application Publication
(10) Pub. No.: US 2013/0253305 A1
(43) Pub. Date: Sep. 26, 2013

(54) System and Method for Imaging of the Vascular Components Using Magnetic Resonance Imaging

(71) Applicant: Ioannis Koktzoglou, Des Plaines, IL (US)

(72) Inventor: Ioannis Koktzoglou, Des Plaines, IL (US)

(21) Appl. No.: 13/782,658

(22) Filed: Mar. 1, 2013

Related U.S. Application Data
(60) Provisional application No. 61/614,575, filed on Mar. 23, 2012.

(51) Int. Cl. A61B 5/055

(52) U.S. Cl. CPC A61B 5/055
USPC 600/410

(57) ABSTRACT

A system and method is provided for acquiring a medical image of a portion of a vascular structure of a subject using a magnetic resonance imaging (MRI) system. A magnetization preparation RF module is applied to a portion of a subject including a vascular structure using the MRI system. A readout procedure is performed to collect image data, wherein the readout procedure includes a phase encoding scheme configured to provide a desired delay time after the application of the magnetization preparation RF module to allow a partial recovery of signal within the vascular structure following application of the magnetization preparation RF module when sampling a central region of k-space during the readout procedure. The image set is reconstructed into an image of the vascular structure wherein blood within the vascular structure is reflected as a gray-blood image.

### CROSS-REFERENCE TO RELATED APPLICATIONS

This application is based on, claims priority to, and incorporates herein by reference, U.S. Provisional Patent Application Ser. No. 61/614,575 filed on Mar. 23, 2012, and entitled “SYSTEM AND METHOD FOR IMAGING OF THE VASCULAR COMPONENTS USING MAGNETIC RESONANCE IMAGING.”

### FIELD OF THE INVENTION

The invention relates to a system and method for performing magnetic resonance imaging and, more particularly, to a system and method for acquiring images using magnetic resonance imaging that allow clinical review of a subject's vascular components, including the blood vessel wall, lumen and intra-wall components.

### BACKGROUND OF THE INVENTION

When a substance such as human tissue is subjected to a uniform magnetic field (polarizing field B₀), the individual magnetic moments of the nuclear spins in the tissue attempt to align with this polarizing field, but precess about it in random order at their characteristic Larmor frequency. Usually the nuclear spins are comprised of hydrogen atoms, but other NMR active nuclei are occasionally used. A net magnetic moment M_{z} is produced in the direction of the polarizing field, but the randomly oriented magnetic components in the perpendicular, or transverse, plane (x-y plane) cancel one another. If, however, the substance, or tissue, is subjected to a magnetic field (excitation field B₁; also referred to as the radiofrequency (RF) field) which is in the x-y plane and which is near the Larmor frequency, the net aligned moment, M_{z}, may be rotated, or “tipped” into the x-y plane to produce a net transverse magnetic moment Mₜ, which is rotating, or spinning, in the x-y plane at the Larmor frequency. The practical value of this phenomenon resides in the signal which is emitted by the excited spins after the excitation field B₁ is terminated. There are a wide variety of measurement sequences in which this nuclear magnetic resonance (“NMR”) phenomenon is exploited.

When utilizing these signals to produce images, magnetic field gradients (Gₓ, G_{y}, and G_{z}) are employed. Typically, the region to be imaged experiences a sequence of measurement cycles in which these gradients vary according to the particular localization method being used. The emitted MR signals are detected using a receiver coil. The MRI signals are then digitized and processed to reconstruct the image using one of many well-known reconstruction techniques.

Magnetic resonance angiography (MRA) and, related imaging techniques, such as perfusion imaging, use the NMR phenomenon to produce images of the human vasculature or physiological performance related to the human vasculature. There are three main categories of techniques for achieving the desired contrast for the purpose of MR angiography. The first general category is typically referred to as contrast enhanced (CE) MRA. The second general category is phase contrast (PC) MRA. The third general category is time-of-flight (TOF) or tagging-based MRA.

Regardless of the particular category of MRA technique utilized, these angiographic techniques typically focus on modifying the contrast associated with the blood flowing within the lumen in the vessel. As such, such imaging techniques often produce so-called “black-blood” or “white-blood” images, which refers to the appearance of the blood as having a “black” (i.e. dark) or “white” (i.e. bright) contrast relative to stationary or tissues that have otherwise not had their associated contrast manipulated using one of the aforementioned techniques.

Many areas of clinical interest focus on the vascular system. For example, atherosclerosis is an inflammatory process in which lipids and calcifications form within the vessel wall to form “plaques.” Atherosclerosis is a major cause of cardiovascular mortality and morbidity in the Western world. As described above, traditional clinical imaging techniques produce “black-blood” or “white-blood” images. “White-blood” images enable the clinician to focus on the degree of luminal impingement by atherosclerotic plaques on the vascular lumen by visualizing an unusual constraint on the vascular lumen. “Black-blood” images are designed to best view the expected appearance of healthy, non-diseased vascular wall. In this regard, traditional MRA techniques provide indirect and incomplete interrogation of vascular disease and vascular plaques by focusing on the appearances of blood and the healthy vascular wall.

Unfortunately, in traditional MRA images, such as “black-blood” or “white-blood” images, the features and boundaries of the vessel lumen, vessel wall, and the ability to accurately discriminate between the vessel lumen and superficial and juxtaluminal intraplaque calcification, as well as other intraplaque components including hemorrhage, are sub-optimally portrayed or generally unavailable to the clinician. For example, the substantial contrast of the black or white blood generally “overpowers” and obscures any subtle contrast of surrounding vascular structures and components. That is, by focusing the enhancing contrast mechanisms in traditional MRA images heavily on either the blood within the vessel or on visualizing healthy vascular wall, direct interrogation of the diseased vascular wall, vascular plaques, and plaque contents through use of a tailored image contrast have generally been foregone in favor of signal properties of the blood proximate to these structures. Accordingly, because the vascular wall characteristics of the in a diseased state and specifically the vascular plaque, plaque contents, and the like, are of greater clinical interest for clinical analysis of conditions such as atherosclerosis and many other vascular conditions, important features can be lost, missed or overpowered in the images produced using traditional MRA techniques.

Furthermore, beyond the constraints on clinical information imposed by such “black-blood” and “white-blood” imaging techniques, the imaging protocols, themselves, present some unfortunately constraints in the clinical setting. For example, black-blood imaging has two major limitations for MRI of the vascular wall. First, black-blood imaging restricts MR data acquisition to a short time window in which the vascular lumen and blood pool is completely suppressed. This restriction limits the temporal efficiency of imaging, or, when this short time window is neglected, introduces substantial apodization in the acquired k-space data and blurring and artifacts in the image space due to the evolving magnetization of the blood pool. Second, black-blood imaging often fails to adequately delineate the vessel lumen from intraplaque calcification due to their similar dark appearance.

A limitation of white-blood imaging techniques (such as 2D and 3D time-of-flight magnetic resonance angiography) for vessel wall imaging is that the extremely hyperintense vessel lumen adjacent to the inner boundary of the vessel wall and the hyperintense regions of perivascular fat adjacent to the outer boundary of the vessel wall severely obscure the boundaries of the relatively hypointense vessel wall. Furthermore, the large flip angles utilized in white-blood imaging largely suppresses the MRI signals in vessel wall. This obscuration of the vessel wall severely limits the ability of white-blood imaging techniques for precise vessel wall area measurement and component characterization of atherosclerotic plaques.

Therefore, it would be desirable to have a system and method for non-invasively producing images that allow clinicians to accurately delineate the shape and boundaries of the vessel lumen and vessel wall and discriminate between the vessel lumen and juxtaluminal intraplaque calcification, as well as depict other intraplaque components including hemorrhage.

### SUMMARY OF THE INVENTION

The present invention overcomes the aforementioned drawbacks by providing a magnetic resonance imaging technique that elicits image contrast in which the blood pool is of a moderate, but distinct, signal intensity from the vessel wall to thereby facilitate unambiguous display and accurate analysis of the vessel wall and associated features and structures. Furthermore, the present invention overcomes the limitations of black-blood imaging by permitting the use of linear phase encode schedules that enhance the imaging point spread function and enable clear separation of superficial and deep-seated intraplaque calcification from the vascular lumen. Also, gray-blood and black-blood image sets (“dual-contrast” imaging) may be simultaneously obtained by modifying the phase-encoding schedule to one that utilizes a mixture of centric and linear phase-encoding ordering.

In accordance with one aspect of the invention, a method is disclosed for acquiring a medical image of a portion of a vascular structure of a subject using a magnetic resonance imaging (MRI) system. The method includes selecting a magnetization preparation radio frequency (RF) module to be applied to a portion of the subject including the vascular structure using the MRI system. The method also includes determining a delay time between an application of the magnetization preparation RF module and a sampling of a central region of k-space during a readout procedure using the MRI system, wherein the delay time is configured to permit partial recovery of signal within the vascular structure following the completion of the magnetization preparation RF module. The method also includes selecting a phase encoding scheme for the readout procedure configured to provide the determined delay time between the application of the magnetization preparation RF module and the sampling of a central region of k-space during the readout procedure. The method further includes performing a pulse sequence using the MRI system to effectuate the selected magnetization preparation RF module, determined delay time, and readout procedure including the selected phase encoding scheme to acquire at least a gray-blood image set and reconstructing the gray-blood image set into a gray-blood image.

In accordance with another aspect of the invention, a magnetic resonance imaging (MRI) system is disclosed that includes a magnet system configured to generate a polarizing magnetic field about at least a portion of a subject arranged in the MRI system, a plurality of gradient coils configured to apply a gradient field to the polarizing magnetic field, and a radio frequency (RF) system configured to apply an excitation field to the subject and acquire MR image data therefrom. A computer system is provided that is programmed to apply a magnetization preparation RF module to a portion of a subject including a vascular structure using the MRI system and perform a readout procedure to collect image data. The readout procedure includes a phase encoding scheme configured to provide a desired delay time after the application of the magnetization preparation RF module to allow a partial recovery of signal within the vascular structure following application the magnetization preparation RF module when the sampling of a central region of k-space during the readout procedure. The computer is further programmed to reconstruct the acquired image set into an image of the vascular structure wherein blood within the vascular structure is reflected as a gray-blood image.

In accordance with yet another aspect of the invention, a method is disclosed for acquiring a medical image of a portion of a vascular structure of a subject using a magnetic resonance imaging (MRI) system. The method includes applying a magnetization preparation RF module to a portion of a subject including a vascular structure using the MRI system and performing a readout procedure to collect image data. The readout procedure includes a phase encoding scheme configured to provide a desired delay time after the application of the magnetization preparation RF module to allow a partial recovery of signal within the vascular structure following application the magnetization preparation RF module when the sampling of a central region of k-space during the readout procedure. The method also includes reconstructing the acquired image set into an image of the vascular structure wherein blood within the vascular structure is reflected as a gray-blood image.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an MRI system for use with the present invention.

FIG. 2 is a schematic representation of a transceiver system for use with the MRI system of FIG. 1.

FIG. 3 is a flow chart of the steps performed in accordance with one exemplary implementation of the present invention.

FIGS. 4A and 4B are diagrams illustrating timing versus phase encoding strategies in accordance with the present invention.

FIG. 5 is a collection of images illustrating black-blood, white-blood, and gray-blood images in side-by-side comparison.

FIG. 6 is a collection of images illustrating black-blood and dual-contrast images in a side-by-side comparison.

### DETAILED DESCRIPTION OF THE INVENTION

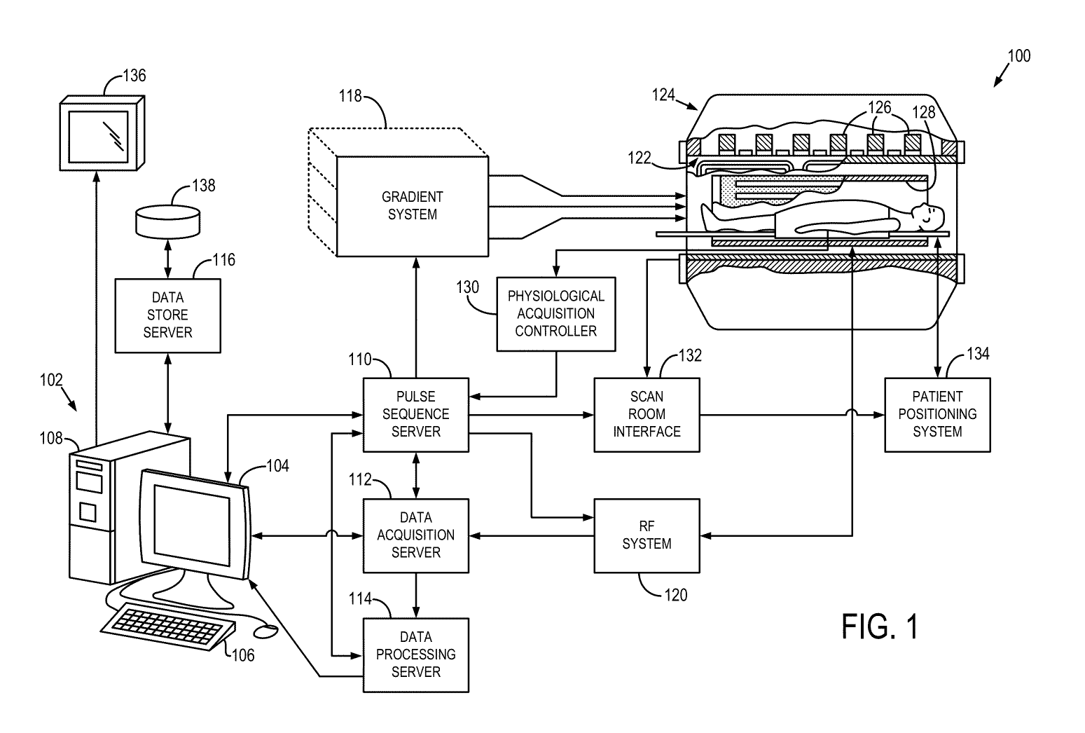

Referring particularly to FIG. 1, an example of a magnetic resonance imaging (“MRI”) system 100 is illustrated. The workstation 102 includes a processor 108, such as a commercially available programmable machine running a commercially available operating system. The workstation 102 provides the operator interface that enables scan prescriptions to be entered into the MRI system 100. The workstation 102 is coupled to four servers: a pulse sequence server 110; a data acquisition server 112; a data processing server 114; and a data store server 116. The workstation 102 and each server 110, 112, 114, and 116 are connected to communicate with each other.

The pulse sequence server 110 functions in response to instructions downloaded from the workstation 102 to operate a gradient system 118 and a radiofrequency (“RF”) system 120. Gradient waveforms necessary to perform the prescribed scan are produced and applied to the gradient system 118, which excites gradient coils in an assembly 122 to produce the magnetic field gradients Gx, Gy, and Gz used for position encoding MR signals. The gradient coil assembly 122 forms part of a magnet assembly 124 that includes a polarizing magnet 126 and a whole-body RF coil 128.

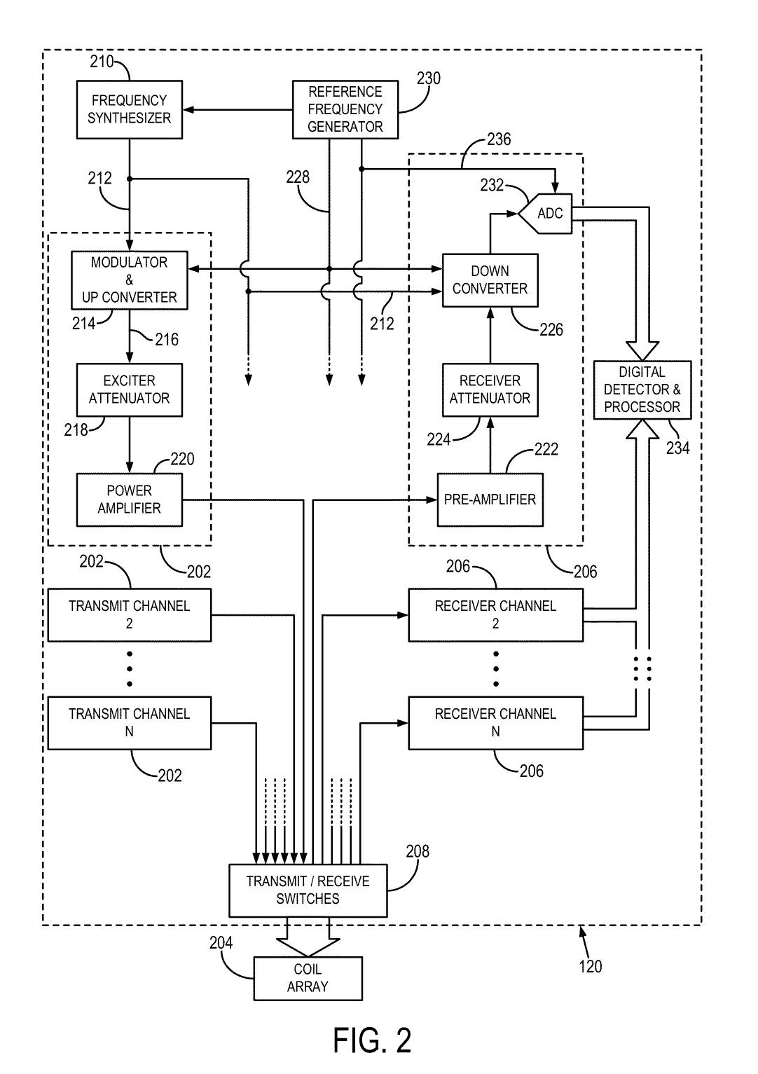

RF waveforms are applied to the RF coil 128, or a separate local coil (not shown in FIG. 2), by the RF system 120 to perform the prescribed magnetic resonance pulse sequence. Responsive MR signals detected by the RF coil 128, or a separate local coil (not shown in FIG. 1), are received by the RF system 120, amplified, demodulated, filtered, and digitized under direction of commands produced by the pulse sequence server 110. The RF system 120 includes an RF transmitter for producing a wide variety of RF pulses used in MR pulse sequences. The RF transmitter is responsive to the scan prescription and direction from the pulse sequence server 110 to produce RF pulses of the desired frequency, phase, and pulse amplitude waveform. The generated RF pulses may be applied to the whole body RF coil 128 or to one or more local coils or coil arrays (not shown in FIG. 1).

The RF system 120 also includes one or more RF receiver channels. Each RF receiver channel includes an RF preamplifier that amplifies the MR signal received by the coil 128 to which it is connected, and a detector that detects and digitizes the I and Q quadrature components of the received MR signal. The magnitude of the received MR signal may thus be determined at any sampled point by the square root of the sum of the squares of the and components:

M=\sqrt{I²+Q²} Eqn. (2);

and the phase of the received MR signal may also be determined:

$$ϕ = {tan}^{- 1} ( \frac{Q}{I} ) . \qquad Wqn . ( 3 )$$

The pulse sequence server 110 also optionally receives patient data from a physiological acquisition controller 130. The controller 130 receives signals from a number of different sensors connected to the patient, such as electrocardiograph (“ECG”) signals from electrodes, or respiratory signals from a bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized MR signal samples produced by the RF system 120 are received by the data acquisition server 112. The data acquisition server 112 operates in response to instructions downloaded from the workstation 102 to receive the real-time MR data and provide buffer storage, such that no data is lost by data overrun. In some scans, the data acquisition server 112 does little more than pass the acquired MR data to the data processor server 114. However, in scans that require information derived from acquired MR data to control the further performance of the scan, the data acquisition server 112 is programmed to produce such information and convey it to the pulse sequence server 110. For example, during prescans, MR data is acquired and used to calibrate the pulse sequence performed by the pulse sequence server 110. Also, navigator signals may be acquired during a scan and used to adjust the operating parameters of the RF system 120 or the gradient system 118, or to control the view order in which k-space is sampled. By way of example, the data acquisition server 112 acquires MR data and processes it in real-time to produce information that may be used to control the scan.

The data processing server 114 receives MR data from the data acquisition server 112 and processes it in accordance with instructions downloaded from the workstation 102. Such processing may include, for example: Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 114 are conveyed back to the workstation 102 where they are stored. Real-time images are stored in a data base memory cache (not shown in FIG. 1), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the workstation 102. The workstation 102 may be used by an operator to archive the images, produce films, or send the images via a network to other facilities.

As shown in FIG. 1, the radiofrequency (“RF”) system 120 may be connected to the whole body RF coil 128, or, as shown in FIG. 2, a transmission section of the RF system 120 may connect to one or more transmit channels 202 of an RF coil array 204 and a receiver section of the RF system 120 may connect to one or more receiver channels 206 of the RF coil array 204. The transmit channels 202 and the receiver channels 206 are connected to the RF coil array 204 by way of one or more transmit/receive (“T/R”) switches 208.

Though illustrated as having multiple transmit channels 202 and multiple receiver channels 206 connected to multiple transmit/receive switches 208, the present invention is not limited to traditional or parallel imaging systems. However, as will be further made apparent below, the dual-contrast imaging technique to be described may particularly benefit from parallel imaging acceleration in the phase-encoding direction.

Also, the receiver channel 206 may also be an assembly of coils separate from the transmit coil array. In such a configuration, the T/R switches 208 are not needed. The transmit coil elements are detuned or otherwise rendered dysfunctional during the receive operation, and the receiver coil elements are similarly detuned or otherwise rendered dysfunctional during operation of the transmit coils. Such detuning may be accomplished with appropriate control logic signals.

Referring particularly to FIG. 2, the RF system 120 includes one or more transmit channels 202 that produce a prescribed RF electromagnetic field. The base, or carrier, frequency of this RF field is produced under control of a frequency synthesizer 210 that receives a set of digital signals from the pulse sequence server 110. These digital signals indicate the frequency, amplitude, and phase of the RF carrier signal produced at an output 212. The RF carrier is applied to a modulator and, if necessary, an up converter 214 where its amplitude and phase is modulated in response to a signal, R(t), also received from the pulse sequence server 110. The signal, R(t), defines the envelope of the RF pulse to be produced and is produced by sequentially reading out a series of stored digital values. These stored digital values may be changed to enable any desired RF pulse envelope to be produced.

The magnitude of the RF pulse produced at output 216 is attenuated by an attenuator circuit 218 that receives a digital command from the pulse sequence server 110. The phase of the RF pulse may also be altered using phase shifters (not shown). The modulated RF pulses are then applied to a power amplifier 220 that drives one element of the RF coil array 204, or several such elements that are electrically coupled. Multiple transmit channels then drive other elements of the multichannel transmit coil array.

The MR signal produced by the subject is picked up by the RF coil array 202 and applied to the inputs of the set of receiver channels 206. A preamplifier 222 in each receiver channel 206 amplifies the signal, which is then attenuated, if necessary, by a receiver attenuator 224 by an amount determined by a digital attenuation signal received from the pulse sequence server 110. The received signal is at or around the Larmor frequency, and this high frequency signal may be down converted in a two step process by a down converter 226. In an example of such a process, the down converter 226 first mixes the MR signal with the carrier signal on line 212 and then mixes the resulting difference signal with a reference signal on line 228 that is produced by a reference frequency generator 230. The MR signal is applied to the input of an analog-to-digital (“ND”) converter 232 that samples and digitizes the analog signal. As an alternative to down conversion of the high frequency signal, the received analog signal can also be detected directly with an appropriately fast analog-to-digital (“A/D”) converter and/or with appropriate undersampling. The sampled and digitized signal may then be applied to a digital detector and signal processor 234 that produces in-phase (I) and quadrature (Q) values corresponding to the received signal. The resulting stream of digitized I and Q values of the received signal are output to the data acquisition server 112. In addition to generating the reference signal on line 228, the reference frequency generator 230 also generates a sampling signal on line 236 that is applied to the A/D converter 232.

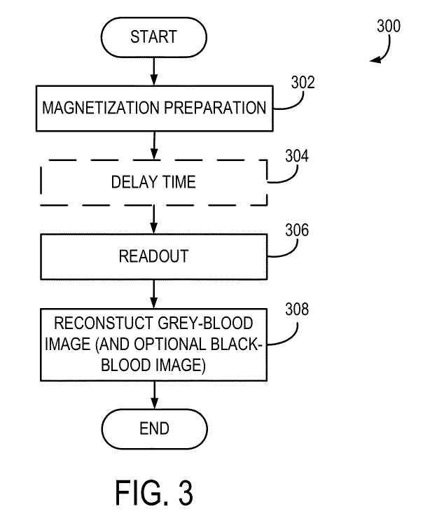

Referring to FIG. 3, a flow chart setting forth the steps of a method 300 for operating an MRI system, such as described above with respect to FIGS. 1 and 2, is provided. The process begins at process block 302 with a flow-suppressing magnetization preparation module. Examples of such flow-suppressing magnetization preparations include motion-sensitized driven equilibrium which are described in Koktzoglou I, Li D. Diffusion-prepared segmented steady-state free precession: Application to 3D black-blood cardiovascular magnetic resonance of the thoracic aorta and carotid artery walls and J Cardiovasc Magn Reson. 2007; 9(1):33-42. PubMed PMID: 17178678 or Balu N, Yarnykh V L, ChuB, Wang J, Hatsukami T, Yuan C. Carotid plaque assessment using fast 3D isotropic resolution black-blood MRI. Magn Reson Med. 2011 March; 65(3):627-37. doi: 10.1002/mrm.22642. Epub 2010 Oct. 12. PubMed PMID: 20941742; PubMed Central PMCID: PMC3042490, both of which are incorporated herein by reference. The flow-suppressing magnetization preparation is applied and is then followed at process block 306 by a gradient-echo based readout utilizing a linear phase-encoding scheme.

As illustrated by intervening process block 304, a delay time is selected between the flow-suppression preparation at process block 302 and the acquisition of central k-space at process bock during a readout at process block 306. Process block 304 is illustrated as optional because it may be embodied as a period of time that elapses between performance of the magnetization preparation at process block 302 and beginning of the readout at process block 306. However, as will be explained, the delay time at process block 304 may be achieved by introducing an “effective delay time” between the magnetization preparation at process block 302 and the sampling of central regions of k-space as part of the readout at process block 306.

The delay time, whether a separate delay time between the magnetization preparation at process block 302 and the commencing readout at process block 306 or an effective delay time between the magnetization preparation at process block 302 and sampling of central regions of k-space as part of the readout at process block 306, may be selected to permit partial recovery of signal within the blood vessel lumen. In the latter case, the delay time may be selected taking into account the k-space sampling pattern so as to delay sampling of central k-space to a desired level, but can be selected so as to not unduly extend the duration of the scan. Because the flow suppressing magnetization-preparation at process block 302 minimally affects the longitudinal magnetization of stationary tissue, the delay time at process block 304 can be selected to allow slight recovery of longitudinal magnetization of the flowing blood pool.

Accordingly, by balancing the k-space sampling pattern of the readout of process block 306 with the delay time of process bock 304, the vascular wall appears hyperintense compared to the blood pool in the image reconstructed at process block 308. This image appearance, which is referred to herein as “gray-blood” image contrast, provides clear and unambiguous delineation of the vessel wall, lumen and intraplaque components such as, but not limited to, calcification and hemorrhage.

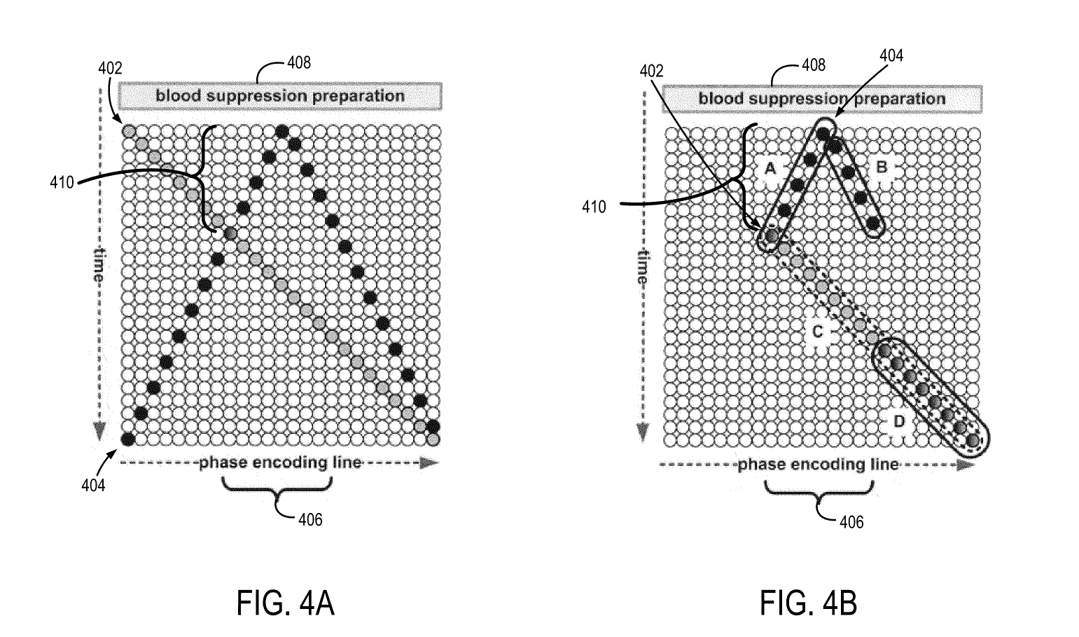

Referring to FIG. 4A, a timing and phase-encoding diagram is provided that contrasts the present gray-blood imaging technique against traditional black-blood imaging techniques. Specifically, FIG. 4A illustrates an exemplary linear phase encoding schedule that may be used with the present invention. In FIG. 4A sampling in accordance with the present invention is illustrated by the gray sampling points 402 in contrast to the traditional black-blood sampling points 404. As shown, conventional black-blood imaging uses a centric phase-encode schedule in which central k-space 406 is acquired shortly after application of the blood suppressing magnetization preparation 408. However, by selecting a gray-blood k-space sampling pattern 402 that, in this case, inherently or “effectively” provides the desired delay time 410 between the application of the flow-suppressing magnetization preparation 408 and the acquisition of the central k-space region 406, a gray-blood phase-encode schedule 402 is performed that yields a desired gray-blood image. Of course, alternative k-space sampling patters are contemplated that can be combined with dedicated delay times 410, for example, when no k-space samplings are performed, to still yield the desired contrast information at the time of sampling the central k-space region 406. However, dedicated delay times serve to extend the overall duration of the scan. In any case, a combination of delay time 410 and a desired gray-blood k-space sampling pattern 402 yields a gray-blood image that provides substantial clinical advantages, for example, when compared to traditional black- or white-blood images.

Specifically, the gray-blood imaging technique of the present invention overcomes the limitations of black-blood imaging by allowing the user to either shorten the acquisition time through the use of longer echo train lengths or to mitigate undesirable signal apodization during the echo train. The latter improves the imaging point spread function and reduces blurring in the reconstructed images. More importantly, gray-blood images provide clinicians with images that substantial improve the ability to identify intraplaque calcification and provide clear separation of juxtaluminal (or superficial) calcification from the vessel lumen, which both appear dark with standard black-blood imaging. With respect to “white-blood” imaging, gray-blood imaging is advantageous in that it provides clear discrimination of the vessel wall from the adjacent vessel lumen, and suppresses image artifacts associated with flowing blood spins.

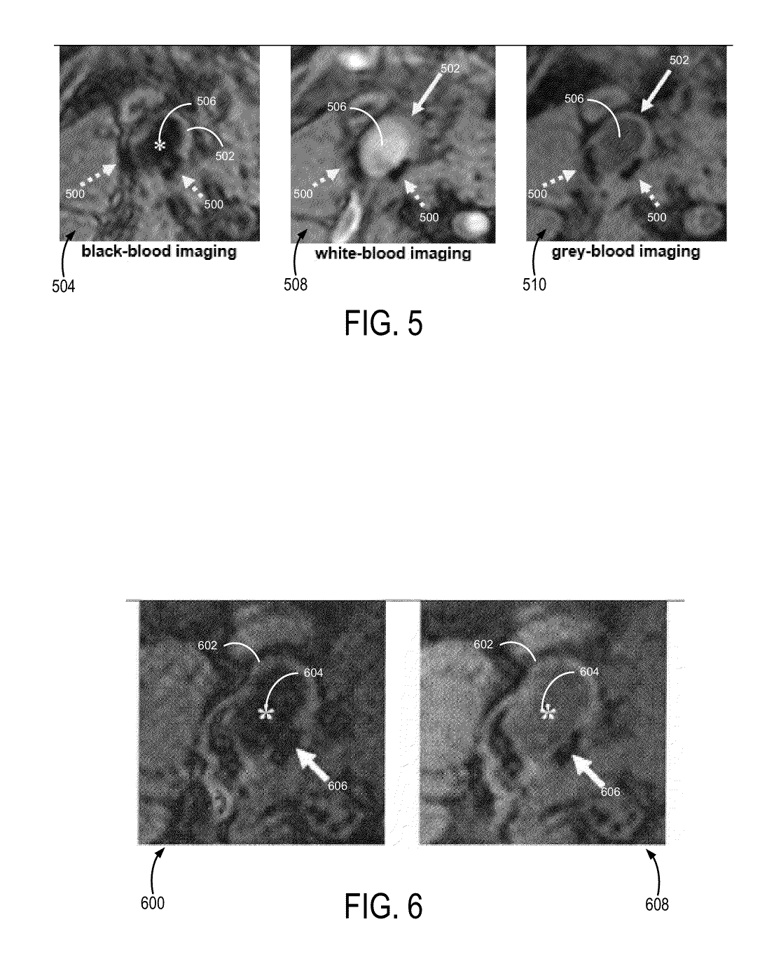

More particularly, FIG. 5 provides a montage of transversal images of the carotid arterial bifurcation of a human subject displaying the some of the clinical advantages of gray-blood image contrast. Calcification 500 within the arterial wall 502 observed with conventional black-blood imaging 504 cannot be clearly separated from the vessel lumen 506. On the white blood-image 508, the calcification 500 is observable, but the vessel wall 502 boundaries cannot be readily identified and distinguished from the vessel lumen 506. However, on the “gray-blood” image 510, calcified regions 500 can clearly be identified and distinguished from the arterial lumen 506 and the arterial wall 502. In addition, the arterial wall 502 can clearly be distinguished from the arterial lumen 506.

It is contemplated that simultaneous gray-blood and black-blood image contrast, or “dual-contrast” imaging, may be obtained by modifying the phase-encoding schedule described above to one that utilizes a mixture of centric and linear phase-encoding ordering. Referring to FIG. 4B, another timing and phase-encoding diagram is provided displaying a hybrid phase encoding schedule permitting the simultaneous acquisition and reconstruction (therefore, the aforementioned “dual contrast imaging”) of a gray-blood image set and a black-blood image set. As described above with respect to FIG. 4A, the gray sampling points 402 are subject to a delay time 410 and focus on the central k-space region. However, whereas FIG. 4A illustrates a black-blood sampling superimposed on the gray-blood sampling, FIG. 4B combines conventional black-blood imaging samples 404 with gray-blood imaging samples 402 after application of the blood suppressing magnetization preparation 408. Specifically, the gray-blood image samples extend along a k-space segment indicated as “C” and the black-blood image samples extend along k-space segments indicated as “A,” “B,” and “D.” In this regard, both the gray-blood and the black-blood image sets can be acquired with no increase in the acquisition time.

It is contemplated that the reconstruction step illustrated with respect to process block 308 of FIG. 3 may utilize techniques such as partial Fourier reconstruction methods, such as homodyne or projection onto convex sets, for example, to recover spatial resolution. So long as a blood suppressing magnetization preparation applied at process block 302 is applied in close temporal proximity to the imaging readout at process block 306, a black-blood image set, such as described above with respect to FIG. 4B, is obtained by reconstructing the lower spatial frequency lines acquired at the beginning of the echo train along with the higher spatial frequency lines acquired later in the echo train. A gray-blood image set is obtained by reconstructing data acquired in the linearly-ordered segment of the echo train. It is also possible to combine oversampled low spatial frequency lines (by standard or weighted signal averaging) to obtain intermediate contrast weightings and to improve signal to noise ratio.

FIG. 6 provides images acquired within the dual-contrast, gray- and black-blood imaging methodology described above. The dual-contrast imaging approach simultaneously provides desirable contrast between the vessel wall and the lumen contrast through reconstruction of a black-blood image set, and enhances the clinician's ability to distinguish and characterize intraplaque constituents through reconstruction of a gray-blood image set. Specifically, FIG. 6 provides image sets of the carotid arteries acquired concurrently with no increase in scan time using the dual-contrast phase encoding schedule of the present invention. The black-blood image 600 readily shows the vessel wall 602 with clinically desirable contrast between the vessel wall 602 and the arterial lumen 604, but obscures the boundary between the superficial calcified intraplaque region 606 and the arterial lumen 604. The gray-blood image 608, on the other hand, precisely shows the extent of vascular calcification 606. Furthermore, image sets acquired with dual-contrast imaging (such as shown in FIG. 6) are also spatially registered which facilitates direct use of both images set for plaque component identification without requiring the use fallible image registration methods.

With respect to gray-blood and dual-contrast imaging, it is contemplated that tailored k-space sampling trajectories, whether Cartesian or non-Cartesian, that frequently or regularly sample the center of k-space can be utilized. For such trajectories, where a plurality of delay times (block 304 of FIG. 3) relative to the acquisition of central k-space can be realized, it is feasible that a plurality of gray blood and black blood images can be generated for vascular wall visualization and plaque component characterization.

The present invention has been described in terms of one or more preferred embodiments, and it should be appreciated that many equivalents, alternatives, variations, and modifications, aside from those expressly stated, are possible and within the scope of the invention.

1. A method for acquiring a medical image of a portion of a vascular structure of a subject using a magnetic resonance imaging (MRI) system, the method comprising:
selecting a magnetization preparation radio frequency (RF) module to be applied to a portion of the subject including the vascular structure using the MRI system;
determining a delay time between an application of the magnetization preparation RF module and a sampling of a central region of k-space during a readout procedure using the MRI system, wherein the delay time is configured to permit partial recovery of signal within the vascular structure following the completion of the magnetization preparation RF module;
selecting a phase encoding scheme for the readout procedure configured to provide the determined delay time between the application of the magnetization preparation RF module and the sampling of a central region of k-space during the readout procedure;
performing a pulse sequence using the MRI system to effectuate the selected magnetization preparation RF module, determined delay time, and readout procedure including the selected phase encoding scheme to acquire at least a gray-blood image set; and
reconstructing the gray-blood image set into a gray-blood image.

2. The method of claim 1 wherein the delay time is performed as an inherent delay time between the magnetization preparation module and the sampling of the central region of k-space by first sampling a periphery of k-space after the magnetization preparation module.

3. The method of claim 1 wherein the delay time is performed as a dedicated passage of time between completion of the magnetization preparation module and initiation of the phase encoding scheme.

4. The method of claim 1 wherein the gray-blood image depicts a vascular wall of the vascular structure as hyperintense compared to a blood pool within the vascular structure.

5. The method of claim 1 wherein the phase-encoding scheme includes a combination of a centric and a linear phase-encoding ordering to collect a black-blood image set and the gray-blood set during the readout procedure.

6. The method of claim 5 further comprising reconstructing the black-blood image set into a black-blood image.

7. The method of claim 1 wherein the readout procedure includes a gradient-echo based readout procedure.

8. The method of claim 1 wherein the readout procedure includes a spin-echo based readout procedure.

9. A magnetic resonance imaging (MRI) system comprising:
a magnet system configured to generate a polarizing magnetic field about at least a portion of a subject arranged in the MRI system;
a plurality of gradient coils configured to apply a gradient field to the polarizing magnetic field;
a radio frequency (RF) system configured to apply an excitation field to the subject and acquire MR image data therefrom;
a computer system programmed to:
apply a magnetization preparation RF module to a portion of a subject including a vascular structure using the MRI system;
perform a readout procedure to collect image data, wherein the readout procedure includes a phase encoding scheme configured to provide a desired delay time after the application of the magnetization preparation RF module to allow a partial recovery of signal within the vascular structure following application of the magnetization preparation RF module when sampling a central region of k-space during the readout procedure;
reconstruct the image set into an image of the vascular structure wherein blood within the vascular structure is reflected as a gray-blood image.

10. The system of claim 9 wherein the desired delay time is an inherent delay time between the magnetization preparation module and the sampling of the central region of k-space by first sampling a periphery of k-space after the magnetization preparation module.

11. The system of claim 9 wherein the delay time is a dedicated passage of time between completion of the magnetization preparation module and performing the readout procedure.

12. The system of claim 9 wherein the gray-blood image depicts a vascular wall of the vascular structure as hyperintense compared to a blood pool within the vascular structure.

13. The system of claim 9 wherein the phase-encoding scheme includes a combination of a centric and a linear phase-encoding ordering to collect a black-blood image set and the gray-blood set during the readout procedure.

14. The system of claim 13 wherein the computer system is further programmed to reconstruct the black-blood image set into a black-blood image.

15. The system of claim 9 wherein the readout procedure includes a gradient-echo based readout procedure.

16. The system of claim 9 wherein the readout procedure includes a spin-echo based readout procedure.

17. A method for acquiring a medical image of a portion of a vascular structure of a subject using a magnetic resonance imaging (MRI) system, the method comprising:
applying a magnetization preparation RF module to a portion of a subject including a vascular structure using the MRI system;
performing a readout procedure to collect image data, wherein the readout procedure includes a phase encoding scheme configured to provide a desired delay time after the application of the magnetization preparation RF module to allow a partial recovery of signal within the vascular structure following application of the magnetization preparation RF module when sampling a central region of k-space during the readout procedure;
reconstructing the image set into an image of the vascular structure wherein blood within the vascular structure is reflected as a gray-blood image.

18. The method of claim 17 wherein the desired delay time is an inherent delay time between the magnetization preparation module and the sampling of the central region of k-space by first sampling a periphery of k-space after the magnetization preparation module.

19. The method of claim 17 wherein the delay time is a dedicated passage of time between completion of the magnetization preparation module and performing the readout procedure.

20. The method of claim 17 wherein the gray-blood image depicts a vascular wall of the vascular structure as hyperintense compared to a blood pool within the vascular structure.

21. The method of claim 17 wherein the phase-encoding scheme includes a combination of a centric and a linear phase-encoding ordering to collect a black-blood image set and the gray-blood set during the readout procedure.

22. The method of claim 21 further comprising reconstructing the black-blood image set into a black-blood image.
